(19) United States
(12) Patent Application Publication
(10) Pub. No.: US 2011/0046735 A1
(43) Pub. Date: Feb. 24, 2011

(54) Patient-Specific Implants

(75) Inventors: Robert Metzger, Wakarusa, IN (US); Brian A. Uthgenannt, Winona Lake, IN (US); Duke A. Fox, Warsaw, IN (US); Brian M. May, Warsaw, IN (US)

Correspondence Address: Harness Dickey & Pierce (Biomet), 5445 Corporate Drive, Suite 200, Troy, MI, 48098, US

(73) Assignee: Biomet Manufacturing Corp., Warsaw, IN (US)

(21) Appl. No.: 12/938,905

(22) Filed: Nov. 3, 2010

Related U.S. Application Data
(63) Continuation-in-part of application No. 12/893,306, filed on Sep. 29, 2010.
Application No. 12/893,306 is a continuation-in-part of application No. 12/888,005, filed on Sep. 22, 2010.
Application No. 12/888,005 is a continuation-in-part of application No. 12/714,023, filed on Feb. 26, 2010.
Application No. 12/714,023 is a continuation-in-part of application No. 12/571,969, filed on Oct. 1, 2009.
Application No. 12/571,969 is a continuation-in-part of application No. 12/486,992, filed on Jun. 18, 2009.
Application No. 12/486,992 is a continuation-in-part of application No. 12/389,901, filed on Feb. 20, 2009.
Application No. 12/389,901 is a continuation-in-part of application No. 12/211,407, filed on Sep. 16, 2008.
Application No. 12/211,407 is a continuation-in-part of application No. 12/039,849, filed on Feb. 29, 2008.
Application No. 12/039,849 is a continuation-in-part of application No. 11/756,057, filed on May 31, 2007.
Application No. 12/039,849 is a continuation-in-part of application No. 11/971,390, filed on Jan. 9, 2008.
Application No. 11/971,390 is a continuation-in-part of application No. 11/363,548, filed on Feb. 27, 2006, now Pat. No. 7,780,672.
Application No. 12/039,849 is a continuation-in-part of application No. 12/025,414, filed on Feb. 4, 2008.
Application No. 12/025,414 is a continuation-in-part of application No. 12/872,663, filed on Aug. 31, 2010.
Application No. 12/872,663 is a continuation-in-part of application No. 12/483,807, filed on Jun. 12, 2009.
Application No. 12/483,807 is a continuation-in-part of application No. 12/371,096, filed on Feb. 13, 2009.
Application No. 12/371,096 is a continuation-in-part of application No. 12/103,824, filed on Apr. 16, 2008.
Application No. 12/103,824 is a continuation-in-part of application No. 12/103,834, filed on Apr. 16, 2008.
(60) Provisional application No. 60/953,620, filed on Aug. 2, 2007.
(60) Provisional application No. 60/947,813, filed on Jul. 3, 2007.
(60) Provisional application No. 60/911,297, filed on Apr. 12, 2007.
(60) Provisional application No. 60/892,349, filed on Mar. 1, 2007.
(60) Provisional application No. 60/812,694, filed on Jun. 9, 2006.
(60) Provisional application No. 60/953,637, filed on Aug. 2, 2007.
(60) Provisional application No. 61/310,752, filed on Mar. 5, 2010.
(60) Provisional application No. 60/912,178, filed on Apr. 17, 2007.
(60) Provisional application No. 60/912,178, filed on Apr. 17, 2007.

(51) Int. Cl. A61F 2/08; A61F 2/38; B23P 13/04

(52) U.S. Cl. 623/14.12; 623/20.32; 29/557

(57) ABSTRACT

An endoprosthetic device includes a monolithic meniscal implant having a three-dimensional patient-specific tibial engagement surface nesting in and complementary to a proximal surface of the proximal tibia of a patient and a femoral articulating surface opposite to the tibial engagement surface for articulating with a femoral condyle of the patient. The meniscal implant includes a first portion and a second remainder portion, wherein the first portion is compressible relative to the second remainder portion.

### CROSS-REFERENCE TO RELATED APPLICATIONS

This application is a continuation-in-part of U.S. application Ser. No. 12/893,306, filed Sep. 29, 2010, which is a continuation-in-part of U.S. application Ser. No. 12/888,005, filed Sep. 22, 2010, which is a continuation-in-part of U.S. application Ser. No. 12/714,023, filed Feb. 26, 2010, which is a continuation-in-part of U.S. application Ser. No. 12/571,969, filed Oct. 1, 2009, which is a continuation-in-part of U.S. application Ser. No. 12/486,992, filed Jun. 18, 2009, and is a continuation-in-part of U.S. application Ser. No. 12/389,901, filed Feb. 20, 2009, which is a continuation-in-part of U.S. application Ser. No. 12/211,407, filed Sep. 16, 2008, which is a continuation-in-part of U.S. application Ser. No. 12/039,849, filed Feb. 29, 2008, which: (1) claims the benefit of U.S. Provisional Application No. 60/953,620, filed on Aug. 2, 2007, U.S. Provisional Application No. 60/947,813, filed on Jul. 3, 2007, U.S. Provisional Application No. 60/911,297, filed on Apr. 12, 2007, and U.S. Provisional Application No. 60/892,349, filed on Mar. 1, 2007; (2) is a continuation-in-part U.S. application Ser. No. 11/756,057, filed on May 31, 2007, which claims the benefit of U.S. Provisional Application No. 60/812,694, filed on Jun. 9, 2006; (3) is a continuation-in-part of U.S. application Ser. No. 11/971,390, filed on Jan. 9, 2008, which is a continuation-in-part of U.S. application Ser. No. 11/363,548, filed on Feb. 27, 2006; and (4) is a continuation-in-part of U.S. application Ser. No. 12/025,414, filed on Feb. 4, 2008, which claims the benefit of U.S. Provisional Application No. 60/953,637, filed on Aug. 2, 2007.

This application is continuation-in-part of U.S. application Ser. No. 12/872,663, filed on Aug. 31, 2010, which claims the benefit of U.S. Provisional Application No. 61/310,752 filed on Mar. 5, 2010.

This application is a continuation-in-part of U.S. application Ser. No. 12/483,807, filed on Jun. 12, 2009, which is a continuation-in-part of U.S. application Ser. No. 12/371,096, filed on Feb. 13, 2009, which is a continuation-in-part of U.S. application Ser. No. 12/103,824, filed on Apr. 16, 2008, which claims the benefit of U.S. Provisional Application No. 60/912,178, filed on Apr. 17, 2007.

This application is also a continuation-in-part of U.S. application Ser. No. 12/103,834, filed on Apr. 16, 2008, which claims the benefit of U.S. Provisional Application No. 60/912,178, filed on Apr. 17, 2007.

The disclosures of the above applications are incorporated herein by reference.

### INTRODUCTION

The present teachings provide a patient-specific endoprosthetic device, such as a meniscal implant, and various customized tibial implants.

### SUMMARY

The present teachings provide an endoprosthetic device that includes a monolithic meniscal implant. The meniscal implant has a three-dimensional patient specific tibial engagement surface nesting in and complementary to a proximal surface of the proximal tibia of a patient and a femoral articulating surface opposite to the tibial engagement surface for articulating with a femoral condyle of the patient. The meniscal implant includes a first portion and a second remainder portion, wherein the first portion is compressible relative to the second remainder portion.

The present teachings provide a meniscal implant that includes a first portion and a second remainder portion. The first portion includes a femoral articulating surface for articulating with a femoral condyle of the patient. The first portion is compressible relative to the second remainder portion. The second remainder portion includes a patient specific tibial engagement surface that is complementary and engageable to a tibial proximal surface of the patient.

The present teachings also provide an endoprosthetic device that includes a tibial bearing having a patient specific profile, a tibial component including a tibial tray having a patient-specific profile, and a tibial stem having a patient-specific orientation relative to the tibial tray.

The present teachings also provide a manufacturing method that includes machining a tibial tray having a patient-specific profile from a tibial tray blank having a greater size that the tibial tray, and machining a customized tibial stem from a stem blank coupled to the tibial tray blank.

Further areas of applicability of the present teachings will become apparent from the description provided hereinafter. It should be understood that the description and specific examples are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIG. 1 is an environmental perspective view of a patient-specific implant according to the present teachings;

FIG. 2 is perspective view of a patient-specific implant according to the present teachings;

FIG. 3 is a perspective view of the patient-specific implant of FIG. 2 illustrating a deformation of the implant in phantom lines;

FIG. 4 is an exploded perspective view of a patient-specific tibial implant;

FIG. 5 is a perspective view of a blank for a patient-specific tibial implant; and

FIG. 6 is a side view of a blank for a patient-specific tibial implant.

### DESCRIPTION OF VARIOUS ASPECTS

The following description is merely exemplary in nature and is in no way intended to limit the present teachings, applications, or uses.

The present teachings generally provide a patient-specific knee endoprosthetic device or implant. The patient-specific implant can be a meniscal replacement or other interpositional knee implant and can be used either with conventional or with patient-specific femoral or tibial implant components prepared with computer-assisted image methods or implanted directly into the patient's natural knee joint without any other implants. Computer modeling for obtaining three dimensional images of the patient's anatomy using MRI or CT scans of the patient's anatomy, the patient-specific prosthesis components, and any associated patient-specific instruments, guides and templates can be designed using various CAD programs and/or software available, for example, by Materialise USA, Ann Arbor, Mich.

Patient-specific implants, alignment guides and other instruments are generally configured to match the anatomy of a specific patient. The patient-specific implants are generally formed using computer modeling based on the patient's three-dimensional (3-D) anatomic image and have an engagement surface that is made to conformingly contact and match a three-dimensional image of the patient's bone surface (with or without cartilage or other soft tissue), by the computer methods discussed above. Various preoperative planning procedures and patient-specific instruments are disclosed in commonly assigned and co-pending U.S. patent application Ser. No. 11/756,057, filed on May 31, 2007; U.S. patent application Ser. No. 12/211,407, filed Sep. 16, 2008; U.S. patent application Ser. No. 11/971,390, filed on Jan. 9, 2008, U.S. patent application Ser. No. 11/363,548, filed on Feb. 27, 2006; and U.S. patent application Ser. No. 12/025,414, filed Feb. 4, 2008. The disclosures of the above applications are incorporated herein by reference.

As disclosed in above-referenced U.S. patent application Ser. No. 11/756,057, filed on May 31, 2007; in the preoperative planning stage for a joint replacement or revision procedure, an MRI scan or a series of CT scans of the relevant anatomy of the patient, such as, for example, the entire leg of the joint to be reconstructed, can be performed at a medical facility or doctor's office. The scan data obtained can be sent to a manufacturer. The scan data can be used to construct a three-dimensional image of the joint and provide an initial implant fitting and alignment in a computer file form or other computer representation. The initial implant fitting and alignment can be obtained using an alignment method, including an alignment protocol used by an individual surgeon.

The outcome of the initial fitting is an initial surgical plan that can be printed or provided in electronic form with corresponding viewing software. The initial surgical plan can be surgeon-specific, when using surgeon-specific alignment protocols. The initial surgical plan, in a computer file form associated with interactive software, can be sent to the surgeon, or other medical practitioner, for review. The surgeon can incrementally manipulate the position of images of implant components in an interactive image of the joint. Additionally, the surgeon can select or modify resection planes, types of implants and orientations of implant insertion. After the surgeon modifies and/or approves the surgical plan, the surgeon can send the final, approved plan to the manufacturer.

After the surgical plan is approved by the surgeon, any patient-specific alignment guides or other instruments can be developed using a CAD program or other imaging software, such as the software provided by Materialise, for example, according to the surgical plan.

Computer instructions of tool paths for machining the patient-specific implants can be generated and stored in a tool path data file. The tool path can be provided as input to a CNC mill or other automated machining system, and the implants can be machined from polymer, ceramic, metal or other suitable material, and sterilized.

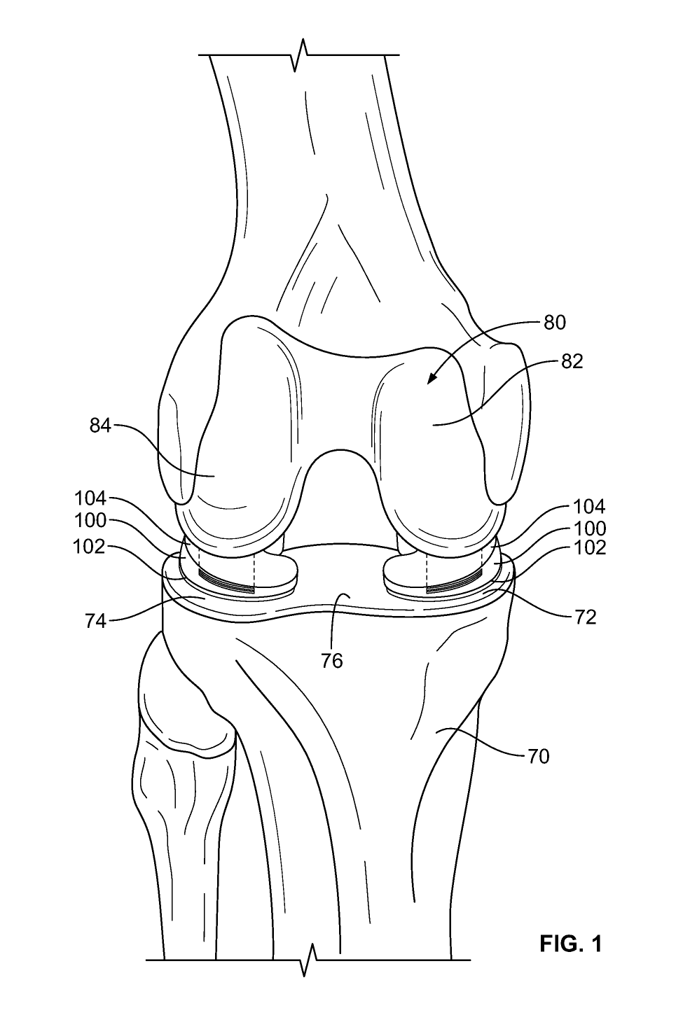

Referring to FIG. 1, an exemplary patient-specific interpositional implant 100 is illustrated in the environment of a knee joint between the distal femur 80 and the proximal tibia 70 of a patient, according to the present teachings. In the exemplary illustration of FIG. 1, the interpositional implant 100 is a meniscal implant 100. Although two such meniscal implants, i.e., medial and lateral, are illustrated between the medial and lateral femoral condyles 82, 84 and the corresponding medial and lateral proximal surfaces 72, 74 of the proximal tibial surface 76 of the tibia 70 of the right (or left) knee, it will be appreciated that only one of the medial and lateral interpositional implants 100 may be used when the other of the medial and lateral menisci need not be replaced.

Referring to FIGS. 1-3, the patient-specific meniscal implant 100 is shaped and sized to correspond to a healthy lateral or medial meniscus of the particular patient. The implant can be generally or overall kidney bean shaped. The patient-specific meniscal implant 100 includes a patient-specific three-dimensional tibial engagement surface 102 that is designed to be complementary and closely conform and nest into and remain substantially in contact with the corresponding proximal surface 72, or 74 of the tibia, based on the pre-preoperative plan for the patient, as discussed above. The patient-specific meniscal implant 100 includes a femoral articulating surface 104 opposite to the tibial engagement surface 102 for articulating with the corresponding natural femoral condyle, 82 or 84. The femoral articulating surface can 104 can also ne patient-specific to match the natural condyle or it can me made to articulate with a femoral implant. Further, the shape and/or perimeter of the meniscal implant 100 can be patient-specific. In one embodiment, the thickness of the meniscal implant 100 can be patient-specific for proper ligament tensioning.

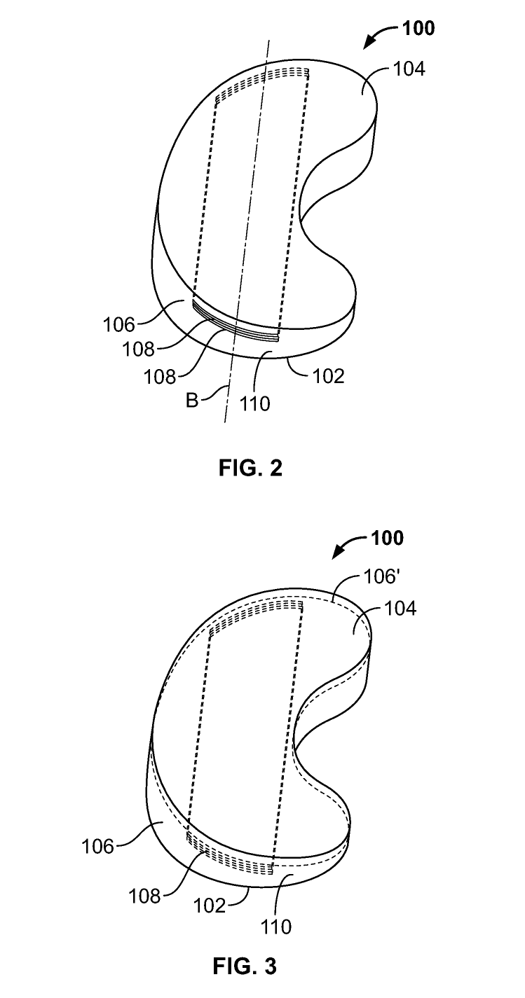

The patient-specific meniscal implant 100 can be made monolithically and unitarily of a biocompatible and relatively rigid material, such as cobalt chrome, for example. The combination of this material and the patient specific tibial engagement surface 102 can help maintain correct positioning of the meniscal implant 100 in the joint, help reduce the degree of dislocation during motion and transmit compressive force with a cushioning effect or shock-absorbing effect. In this respect, the patient specific tibial engagement surface 102 can remain substantially engaged and in contact with the proximal surface of the tibia during motion. Additionally, an upper or superior portion 106 of the patient-specific meniscal implant 100 can be made compliant to further reduce the risk of dislocation, reduce contact stresses by providing better conformance during articulation relative to the femoral condyles and transmit compressive forces with a cushioning effect. Specifically, a first or superior portion 106 of the patient-specific meniscal implant 100 can be made compliant or flexible and compressible by creating a series or an array of cutouts or notches or weakened areas or slits 108, using for example Electrical Discharge Machining (EDM) or spark erosion. The slits 108 lie completely under and do not break or extend through the articulating surface 104, which, therefore, remains intact and can be highly polished for articulation with a femoral implant or with the natural femoral condyles of the patient. The first portion 106 that includes the slits 108 can extend to about one quarter of the thickness of the meniscal implant 100. The slits 108 can be arranged either in an anterior-posterior direction along an anterior-posterior axis B, as shown in FIG. 2, or in a medial-lateral direction along an axis perpendicular to axis B (not shown). The slits 108 can be parallel and separate or interconnected in a zigzag pattern. The slits 108 can also be curved. The first portion 106 includes and lies directly adjacent and under the articulating surface 104 and extends partway toward but not completely through the entire thickness of the meniscal implant 100. Accordingly, a second inferior or remainder portion 110 including and adjacent to the tibial engagement surface 102 is not deformable or not compressible relative to the first portion 106. The EDM can be of the wire type and can be numerically controlled. The array of weakened areas 108 imparts flexibility, resilience, compliance and elastic or recoverable deformability to the first portion 106 of the meniscal implant 100, such that the first portion 106 can move, deform or be compressed to a second configuration 106′ (schematic representation) relative to the second remainder portion 110 of the patient-specific meniscal implant 100. The slits 108 can impart a compressibility of about 30% of the total height of the meniscal implant 100 in a superior-inferior direction and transmit and distribute compressive loads evenly along the meniscal implant 100.

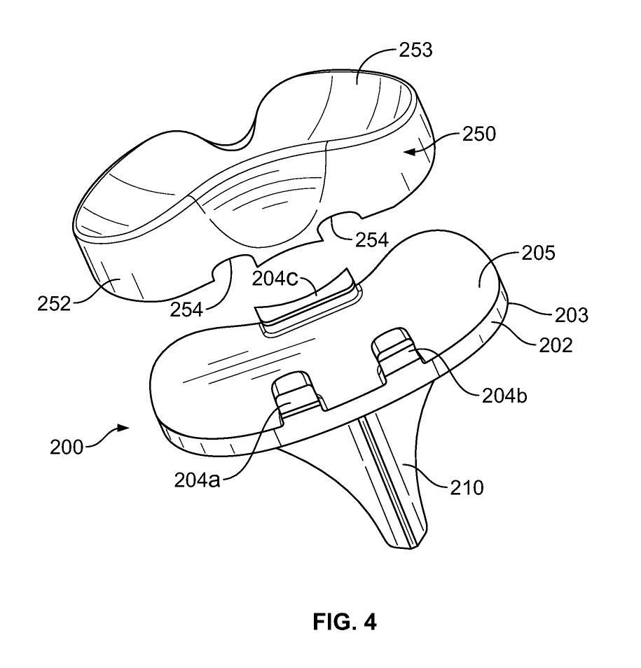

Referring to FIGS. 4-6, customized tibial implants are illustrated. The tibial implants can be fully customized/patient-specific portions, semi-custom portions and/or off the shelf portions. For example, the tibial implants can include a tibial bearing 250 and a tibial component 200 having a tibial tray 205 and a tibial stem 210. The tibial bearing 250 and the tibial tray 204 can have respective patient-specific perimeters/peripheries or profiles 252, 202 that match and conform to the patient's anatomy based on the three-dimensional image data of the patient's bone, as disclosed in commonly assigned U.S. Patent Application 2008/0262624, published Oct. 23, 2008. The tibial bearing 250 can also have a patient-specific thickness and a patient-specific articulating surface 253 for articulation with the natural femoral condyles. The tibial component 200 can have standard, i.e., non patient-specific locking tabs or other locking features 204a, 204b, 204c that can engage corresponding grooves or recesses or other formations 254 of standard or customized bearings 250. In this respect, the bearing 250 and the tibial component 200 can combine patient-specific and standard (non patient-specific) features and elements and can be characterized as semi-custom implants. In some embodiments, the tibial tray 204 can have a patient-specific bone-engaging surface 203, a patient-specific periphery 202 and a patient-specific stem 210 to conform the specific contouring of the patient.

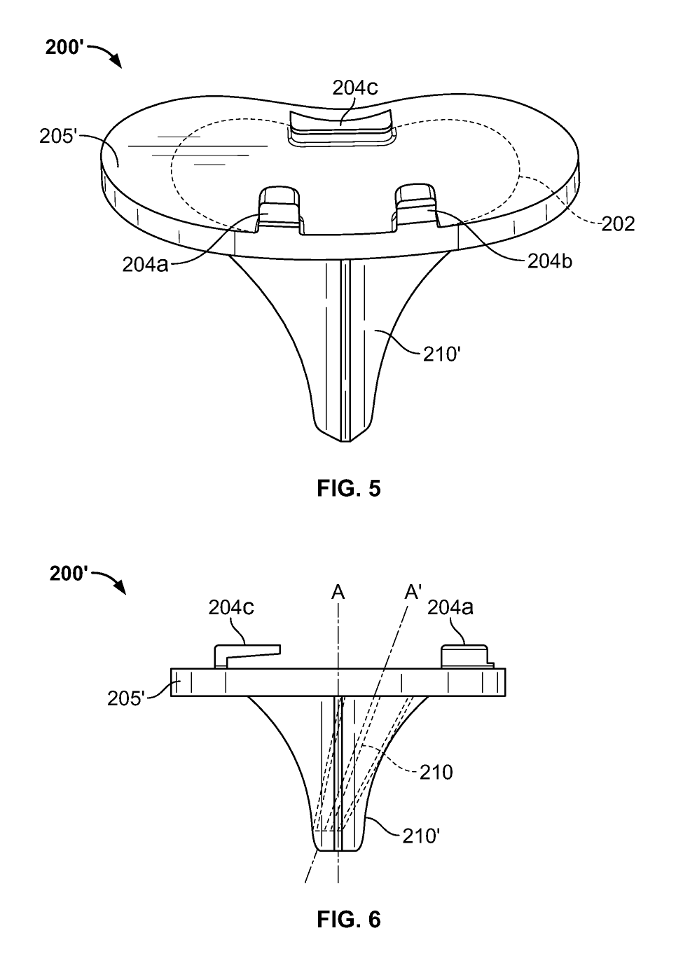

Referring to FIGS. 5 and 6, the customized tibial component 200 can be prepared from a generic tibial blank or generic tibial casting 200′ from which customized tibial components of various sizes can be machined using numerical control with patient-specific data. The generic tibial blank 200′ can be made to include the final standardized locking tabs 204a, 204b, 204c, but can have a tray blank 205′ of a bigger size from which the tray 205 with the patient specific periphery or profile 202 can be prepared by machining or other methods. When the tibial tray 205 is machined from the tray blank 205′, the locking tabs 204a, 204b, 204c are incorporated intact into the tibial tray 205. In one embodiment, one of the locking tabs 204c can be inbound relative to the tray blank 205′.

Alternatively, in other embodiments, the locking tabs 204a, 2004b, 204c may otherwise be shaped or trimmed or customized for non-standard or patient-specific grooves 254 and can also be configured for used with floating bearings.

Similarly, the tibial stem 210 can be customized out of a bigger stem blank 210′ that can accommodate a patient-specific stem orientation along a patient-specific axis A′ relative to a standardized axis A that is perpendicular to the tray blank 205′ of the tibial blank 200′. The patient-specific axis A′ can be oriented at a customized posterior slope, for example. Additionally, the patient-specific stem 210 can be designed from the stem blank 210′ to have shape and size, including thickness and length, designed for the particular patient. The patient-specific stem 210 can be built-in or modularly coupled to the tibial tray 205 and can be machined to a desired type, such as I-beam type, cruciate fin type, or splined, tapered stem type of stem. Additionally, the patient-specific stem 210 can be customized for a patient specific orientation relative to the anterior-posterior and/or medial-lateral directions.

The tray blank 205′ and or the stem blank 210′ can be made of porous metal, such as porous titanium, or of a solid metal, such as titanium.

The foregoing discussion discloses and describes merely exemplary arrangements of the present teachings. Furthermore, the mixing and matching of features, elements and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the present teachings without departing from the essential scope thereof. One skilled in the art will readily recognize from such discussion, and from the accompanying drawings and claims, that various changes, modifications and variations can be made therein without departing from the spirit and scope of the present teachings as defined in the following claims.

What is claimed is:

1. An endoprosthetic device comprising:
a monolithic meniscal implant having a three dimensional patient-specific tibial engagement surface nesting in and complementary to a proximal surface of the proximal tibia of a patient, a femoral articulating surface opposite to the tibial engagement surface for articulating with a femoral condyle of the patient, the meniscal implant including a first portion and a second remainder portion, wherein the first portion is compressible relative to the second remainder portion.

2. The endoprosthetic device of claim 1, wherein the first portion includes a plurality of cutouts providing resiliency to the first portion.

3. The endoprosthetic device of claim 2, wherein the meniscal implant comprises cobalt chrome.

4. The endoprosthetic device of claim 2, wherein the cutouts are made by electrical discharge machining.

5. The endoprosthetic device of claim 4, wherein the cutouts are slits that impart about thirty percent compressibility to the meniscal implant in a superior-inferior direction.

6. The endoprosthetic device of claim 1, wherein the plurality of cutouts includes slits extending in an anterior-posterior direction.

7. The endoprosthetic device of claim 6, wherein the plurality of cutouts includes slits parallel to and under to the femoral articulating surface.

8. The endoprosthetic device of claim 6, wherein the plurality of slits extend to about one quarter of the meniscal implant in a superior to inferior direction.

9. The endoprosthetic device of claim 1, further comprising a tibial bearing having a patient specific profile.

10. The endoprosthetic device of claim 9, wherein the tibial bearing has a patient-specific articulating surface.

11. The endoprosthetic device of claim 9, further comprising a tibial tray having a patient-specific profile.

12. The endoprosthetic device of claim 11, wherein the tibial tray has a patient-specific bone engaging surface.

13. The endoprosthetic device of claim 12, further comprising a tibial stem coupled to the tibial tray, the tibial stem having a patient-specific orientation relative to the tibial tray.

14. The endoprosthetic device of claim 11, wherein the tibial tray is machined from a tray blank capable of accommodating different-size tibial trays, the tibial blank having a plurality of locking tabs engageable with corresponding grooves of the tibial bearing.

15. An endoprosthetic device comprising:
a meniscal implant including a first portion and a second remainder portion, the first portion including a femoral articulating surface for articulating with a femoral condyle of the patient, the first portion being compressible relative to the second remainder portion, the second remainder portion including a patient specific tibial engagement surface, the tibial engagement surface complementary and engageable to a tibial proximal surface of the patient.

16. The endoprosthetic device of claim 13, wherein the first portion includes a plurality of cutouts providing resiliency and compressibility to the first portion.

17. The endoprosthetic device of claim 16, wherein the cut-outs are slits providing about thirty percent compressibility relative to the entire height of the meniscal implant,

18. An endoprosthetic device comprising:
a tibial bearing having a patient specific profile;
a tibial component including a tibial tray having a patient-specific profile; and
a tibial stem having a patient-specific orientation relative to the tibial tray.

19. The endoprosthetic device of claim 18, wherein the tibial tray is machined from a tray blank capable of accommodating different-size tibial trays, the tibial blank having a plurality of locking tabs engageable with corresponding grooves of a tibial bearing.

20. The endoprosthetic device of claim 18, wherein the locking tabs are not patient-specific.

21. The endoprosthetic device of claim 18, wherein the tibial bearing has a patient-specific articulating surface.

22. A manufacturing method comprising:
machining a tibial tray having a patient-specific profile from a tibial tray blank having a greater size that the tibial tray; and
machining a customized tibial stem from a stem blank coupled to the tibial tray blank.

23. The method of claim 22, further comprising machining the tibial stem to have a patient-specific orientation relative to the tibial tray.

24. The method of claim 22, further comprising machining the tibial stem to have a patient-specific length.

25. The method of claim 22, further comprising machining the tibial stem to have a patient-specific shape.

26. The manufacturing method of claim 22, wherein machining a tibial tray having a patient-specific profile form a tibial tray blank includes incorporating a plurality of locking tabs from tibial tray blank to the tibial tray.

27. The manufacturing method of claim 26, further comprising customizing the locking tabs.
